SEQUENCE LISTING (6 sequences)

SEQ ID NO: 1 - DNA, 23 bp, Artificial Sequence
  synthetic oligonucleotide
tctcaaatcagaatgggacaggt

SEQ ID NO: 2 - DNA, 20 bp, Artificial Sequence
  synthetic oligonucleotide
tgagaaccgtttgtgttgcg

SEQ ID NO: 3 - DNA, 27 bp, Artificial Sequence
  synthetic oligonucleotide
ttccattacaagcatgatcgggagggt

SEQ ID NO: 4 - DNA, 24 bp, Artificial Sequence
  synthetic oligonucleotide
gagatcttgtacattcgcaccaat

SEQ ID NO: 5 - DNA, 26 bp, Artificial Sequence
  synthetic oligonucleotide
ttaactgagaagaggagttcgatgct

SEQ ID NO: 6 - DNA, 26 bp, Artificial Sequence
  synthetic oligonucleotide
cttcagttccttccattcccttggnn

(19) United States
(12) Patent Application Publication
(10) Pub. No.: US 2009/0280112 A1
(43) Pub. Date: Nov. 12, 2009

(54) INHIBITORY EFFECTS OF NORDIHYDROGUAIARETIC ACID (NDGA) ON THE IGF-1 RECEPTOR AND ANDROGEN DEPENDENT GROWTH OF LAPC-4 PROSTATE CANCER CELLS

(75) Inventors: IRA D. Goldfine, Belvedere, CA (US); Jack F. Youngren, San Francisco, CA (US); Michael J. Campbell, Woodside, CA (US); Betty A. Maddux, San Francisco, CA (US); John A. Kerner, San Francisco, CA (US); Charles J. Ryan, Mill Vally, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Regents of the University of California

(21) Appl. No.: 12/434,071

(22) Filed: May 1, 2009

Related U.S. Application Data
(60) Provisional application No. 61/050,561, filed on May 5, 2008.

(51) Int. Cl. A61K 39/395; A61P 35/00; A61K 31/7088; A61K 31/56; A61K 31/496; A61K 31/675; A61K 31/196; A61K 31/506

(52) U.S. Cl. 424/130.1; 514/44 A; 514/171; 514/254.07; 514/110; 514/567; 514/252.19

(57) ABSTRACT

Disclosed herein are methods and compositions for the treatment of prostate cancer with an IGF-1 receptor kinase inhibitor. Methods are also provided for the treatment of prostate cancer by identifying a level of IGF-1 receptor expression and making a decision whether to treat with an IGF-1 receptor kinase inhibitor.

### CROSS-REFERENCE

This application claims the benefit of U.S. Provisional Application No. 61/050,561, filed May 5, 2008, which application is incorporated herein by reference.

### GOVERNMENT RIGHTS

This invention was made with government support under federal grant nos. NIH/K23CA115775 awarded by the National Institutes of Health. The United States Government has certain rights in this invention.

### FIELD OF THE INVENTION

The invention relates generally to methods of treating cancer and more particularly to treating individuals afflicted with prostate cancer with a formulation comprised of an inhibitor of IGF-1 receptors such as NDGA. The method includes contacting the patients prosthetic cancer cells with a formulation of the invention in sufficient amount and for sufficient period of time so as to have a therapeutic result in treating the cancer cells.

### BACKGROUND OF THE INVENTION

Prostate cancer is the most common cancer in men, accounting for over 33% of all new cancer cases each year. Although prostate cancer has a relatively low mortality rate, it is the third leading cause of cancer death in men in the United States, with about 27,350 estimated deaths in 2006. The incidence of prostate cancer also increases with age, with an increase of over 1000% for men over 65 years of age.

One of the hallmarks of prostate cancer is the tumor's sensitivity to androgen stimulation of growth, a process that relies on a variety of ligand directed and co-stimulatory mechanisms. See Nieto et al., Scher et al. Others have hypothesized that certain non-androgen signaling mechanisms within prostate cancer cells may also regulate tumor cell proliferation, many of which may be implicated in the emergence of progressive disease in a testosterone-depleted milieu. Certain of these non-androgen mechanisms appear to include cell surface tyrosine kinases including the insulin like growth factor-1 receptor (IGF-1R). See Baserga; Burfeind et al.; Nickerson et al. and Pollak. However, the contribution, if any, of each pathway towards the development of human prostate cancer is still unknown.

Current treatment of benign or localized prostatic cancer comprises removal of the cancerous organ and/or localized radiotherapy, with fairly high success levels of eradication or elimination of the tumor. In metastatic patients, treatment may also involve androgen-deprivation therapy, which may initially be effective and result in disease remission. Androgen-deprivation therapy in males, which targets levels of mainly testosterone and dihydrotestosterone, may comprise ligand deprivation, including castration (orchiectomy), and/or anti-androgen treatment. Androgen-deprivation therapy may result in serious side effects that often overshadow the effects of the prostate cancer itself, including loss of potency and sexual libido associated with orchiectomy, as well as development of osteoporosis, anemia and liver dysfunction.

Although initially effective, prostate tumors may recur within 2-3 years after androgen-deprivation therapy, at which stage such therapy may be ineffective. These recurrent tumors, in addition, do not respond to conventional therapies, and are often considered incurable. Moreover, patients treated for benign or localized prostatic cancer through surgical and/or radiotherapy means may also later develop invasive or micrometastatic growth of prostatic cancer, subjecting the individual to androgen-deprivation therapy and its attendant side effects.

Accordingly, an urgent need remains for effective treatment of prostate cancer growth and metastasis.

### SUMMARY OF THE INVENTION

The invention includes a formulation which is manufactured for use in treating a human afflicted with prostate cancer which formulation is comprised of a pharmaceutically acceptable carrier and an IGF-1 receptor inhibitor. The formulation may be specifically manufactured for use in treating an individual afflicted with androgen-nonresponsive prostate cancer or an individual afflicted with androgen-responsive prostate cancer.

The formulation may be manufactured for use in treating humans wherein the IGF-1 receptor inhibitor is selected from the group consisting of a small molecule inhibitor, an antisense oligonucleotide or an antibody. Still further, the small molecule inhibitor may be selected from the group consisting of an NDGA, NVP-AEW541 and picropodophyllin and wherein the formulation is manufactured for use in treating a type of prostate cancer selected from the group consisting of benign, localized and metastatic.

The formulation may further comprise an androgenic hormone blocking agent such as the agent selected from the group consisting of an LHRH analog, an LHRH antagonist, an antiandrogen, an estrogen, and ketoconazole.

The formulation may include an anticancer agent which is a chemotherapeutic or a cytotoxic agent wherein the chemotherapeutic agent may be cyclophosphamide and wherein the cytotoxic agent is chlorambucil.

The formulation may include an agent that inhibits nonreceptor-tyrosine kinases wherein the agent is selected from the group consisting of dasatinib, AZDO530, AP23846, PP2 and UCS15A.

In a specific embodiment of the invention the formulation is manufactured for use in treating a human afflicted with prostate cancer and is comprised of NDGA which may be present in an NDGA solvent such as DMSO and may further comprise an adrenal androgen inhibitor.

A method of treating prostate cancer in a human is disclosed which method includes diagnosing a human patient as having prostate cancer and then determining if the prostate cancer is responsive to androgen therapy. Thereafter the patient is treated by administering a therapeutically effective amount of a formulation comprising a pharmaceutically acceptable carrier and an IGF-1 receptor inhibitor and thereafter allowing the formulation to act on cancer cells in the human and treat the prostate cancer.

In another aspect of the invention the patient is further treated by administering an androgenic hormone blocking agent and allowing the agent to treat the prostate cancer wherein the agent may be any agent typically used in such therapy including an agent selected from the group consisting of an LHRH analog, an LHRH antagonist, an antiandrogen, an estrogen, and ketoconazole.

The method of treatment may further include administering a cyclophosphamide to a human and allowing the cyclophosphamide to treat the prostate cancer and may further include administering chlorambucil to the patient and allowing such to treat the cancer.

Provided herein are methods of treating an individual afflicted with prostate cancer, wherein the individual is treated with a formulation comprising at least one inhibitor to IGF-1 receptor. The inhibitor to IGF-1 receptor may be a small molecule inhibitor, an antisense oligonucleotide or an antibody. Alternatively, the inhibitor may inhibit tyrosine-kinase or autophosphorylation activity of the IGF-1 receptor. By way of example only, the inhibitor to IGF-1 receptor may be chosen from the group consisting of nordihydroguiaretic acid (NDGA), NVP-AEW541 and picropodophyllin and combinations thereof. Alternatively, the IGF-1 receptor inhibitor may include tyrphostin AG-538 or IGF-1R inhibitors disclosed in U.S. application Ser. No. 10/814,199 (US 2004/0209930), including IGF-1R inhibitors described in U.S. Pat. No. 7,081,454; U.S. Pat. No. 7,189,716; U.S. Pat. No. 7,232,826, U.S. Pat. No. 6,337,338; WO 00/35455; WO 02/102804; WO 02/092599; WO 03/024967; WO 03/035619; WO 03/035616; WO 03/018022 all incorporated here by reference to disclose and describe such inhibitors.

The prostate cancer treated via the present invention may be benign or localized or alternatively the prostate cancer may be metastatic or invasive. The method of the invention may be used to treat an individual afflicted with prostate cancer that does not undergo concomitant androgen-deprivation therapy. In yet other embodiments, the individual afflicted with prostate cancer does undergo concomitant androgen-deprivation therapy alongside the IGF-1 receptor therapy provided herein.

In yet other embodiments the invention may further comprise providing at least one anti-cancer, cytotoxic or chemotherapeutic agent with the formulation. The cytotoxic or chemotherapeutic agents may include alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6 MP), 6-thiocguanine (6TG), cytarabine (Ara-C), 5-fluorouracil (5-FU), capecitabine (e.g. XELODA.®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

In another embodiment, the formulation may further comprise an agent that inhibits nonreceptor-tyrosine kinases. By way of example only, the inhibitors to nonreceptor tyrosine kinases may be chosen from the group consisting of dasatinib, AZDO530, AP23846, PP2 or UCS15A and combinations thereof.

Also provided herein are methods of treating an individual afflicted with prostate cancer, wherein the individual is treated with a formulation comprising NDGA. In some embodiments, the prostate cancer may be benign or localized. In other embodiments the prostate cancer may be metastatic or invasive. In yet other embodiments, the individual afflicted with prostate cancer does not undergo concomitant androgen-deprivation therapy. In yet other embodiments, the individual afflicted with prostate cancer does undergo concomitant androgen-deprivation therapy alongside the IGF-1 receptor therapy provided herein. In addition, other embodiments may include in the formulation provided to the individual afflicted with prostate cancer an anti-cancer agent and/or an agent that inhibits nonreceptor tyrosine kinases. Such agents may include by way of example only dasatinib, AZDO530, AP23846, PP2 or UCS15A and combinations thereof.

Provided herein are also methods for treating an individual afflicted with androgen-responsive prostate cancer comprising treating the individual afflicted with prostate cancer with a formulation comprising an IGF-1 receptor inhibitor and an anti-cancer agent. In some embodiments, the IGF-1 receptor inhibitor may be a small molecule inhibitor, an antisense oligonucleotide or an antibody. By way of example only, the small molecule inhibitor may be chosen from the group consisting of NDGA, NVP-AEW541 or picropodophyllin and combinations thereof.

Additionally provided herein are methods for treating an individual afflicted with androgen-responsive prostate cancer comprising first identifying a level of IGF-1 receptor expression in a sample the individual, and making a decision based on the level of IGF-1 receptor expression whether to treat said individual with an inhibitor to IGF-1 receptor. In some embodiments, the level of IGF-1 receptor expression is increased as compared to a baseline level of IGF-1 receptor expression.

In some embodiments the sample may be selected from the group consisting of tissue, plasma, blood, serum, hair, cell, organ, sputum, saliva, semen, prostatic fluid and pre-ejaculate.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 includes graphs (a), (b) and (c) which show the effect of dihydrotestoneron (DHT) and other androgens on the proliferation of a prostate cancer cell line, LAPC-4.

FIG. 2 includes graphs (a) and (b) which show the effect of IGF-1 receptor inhibtors, including NVP-AEW541 and picropodophyllotoxin (PPP) on DHT-induced cell proliferation.

FIG. 3 includes graphs (a) and (b) which show the effect of NDGA on inhibiting DHT-induced prostate cancer cell proliferation.

FIG. 4 includes graphs (a) and (b) which show the effect of NDGA on inhibiting IGF-1 receptor autophosphorylation.

FIG. 5 shows Western blots (a), (c) and (d) and graphs (b) and (e) which show the effect of DHT to increase the expression of IGF-1 receptor, and the inhibition of expression of IGF-1 receptor by NDGA.

FIG. 6 includes graphs (a) and (b) which show the effect of NDGA to inhibit DHT-induced IGF-1 receptor gene expression, but not androgen-receptor conformation.

### DETAILED DESCRIPTION OF THE INVENTION

Before the present methods and formulations for treatments are described, it is to be understood that this invention is not limited to particular methods, formulations or uses described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a molecule” includes a plurality of such molecules and reference to “the administration” includes reference to one or more administrations and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Provided herein are methods of treating an individual afflicted with prostate cancer comprising treatment with at least one tyrosine kinase inhibitor.

In certain embodiments,

The IGF-1R and its ligands, IGF-1 and IGF-2, play a key role in regulating growth, resistance to apoptosis, and invasion in a variety of human cancers (7-10). A number of studies have established a role for the IGF system in prostate cancer. First, clinical and epidemiological data indicate that elevated serum IGF-1 levels are a risk factor for prostate cancer (11, 12). Second, the IGFs increase the growth of prostate cancers in cultured cells (13, 14). Third, abrogation of the IGF-1R via anti-sense suppresses growth and invasion by rat prostate cells in vivo (4). Further, the progression of some androgen sensitive cell lives to androgen independent growth in xenografts, is accompanied by an increased expression of both IGF-1 and IGF-1R.

Meso-nordihydroguaiaretic acid (NDGA), a butanediol, is a compound isolated from Larrea tridentata, more commonly known as chaparral or the creosote bush L. tridentata grows in the southwestern United States and Mexico, and extracts of the leaf and/or stem have been taken orally by the Pima Indians and other cultures in these regions to treat various conditions (15). Prior studies from our laboratory have demonstrated that purified NDGA inhibits the IGF-1R tyrosine kinase (16-18). In breast cancer and neuroblastoma cells, NDGA both inhibits growth in tissue culture and reduces tumorigenesis (17, 19) in animals. we have recently reported that this receptor is expressed in nearly all prostate cancers and metastases (20). Accordingly, the IGF-1R is a potential target in these cancers.

In the present study, the effects of NDGA on the growth and proliferation of prostate cancer cells stimulated with androgen are evaluated. The human prostate cancer cell line, LAPC-4, was utilized because of the absence of mutations in either the androgen receptor (AR) or PTEN, and its sensitivity to androgen stimulation of proliferation (21, 22). We now report that, in LAPC-4 cells grown in tissue culture, NDGA attenuates growth in concert with both direct inhibition of the IGF-1R tyrosine kinase and inhibition of androgen-stimulated expression of the IGF-1R protein.

### CERTAIN DEFINITIONS

Unless indicated otherwise, the following terms have the following meanings when used herein and in the appended claims.

As used herein, “expression” refers to one or more of the following events: (1) production of an RNA template from a DNA sequence (e.g., by transcription) within a cell; (2) processing of an RNA transcript (e.g., by splicing, editing, 5′ cap formation, and/or 3′ end formation) within a cell; (3) translation of an RNA into a polypeptide or protein within a cell; (4) post-translational modification of a polypeptide or protein within a cell; (5) presentation of a polypeptide or protein on the cell surface; (6) secretion or release of a polypeptide or protein from a cell.

As used herein, “over-expression”, refers to a higher level of expression when compared to the endogenous level of expression of an identical polypeptide or protein within the same cell. In some embodiments a higher level of expression comprises 2% to 200% higher. In some embodiments a higher level of expression comprises 2-fold to 1000-fold higher. In some embodiments a higher level of expression comprises 2-fold to 1000-fold higher. In some embodiments a higher level of expression comprises 2-fold to 10,000-fold higher. In some embodiments a higher level of expression comprises a detectable level of expression when compared to a previous undetectable level of expression. In some embodiments “over-expression” refers to any detectable level of expression of an exogenous polypeptide or protein.

As used herein, “over-expression of IGF-1 receptor” refers to over-expression of an IGF-1 receptor polypeptide or an IGF-1 receptor polypeptide fused to another polypeptide. In some embodiments “over-expression of IGF-1 receptor” refers to over-expression of IGF-1 receptor mRNA or nucleotide encoding IGF-1 receptor. In some embodiments “over-expression of IGF-1 receptor” refers to over-expression of a fragment of an IGF-1 receptor polypeptide.

The terms “polypeptide”, peptide” and “protein” are used interchangeably herein to refer to a polymer of amino acid residues. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to agents that have the same basic chemical structure as a naturally occurring amino acid, i.e., an a carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids are referred to herein by either their commonly known three letter symbols or by the one-letter symbols recommended by the IUPAC-IUB Biochemical Nomenclature Commission. Nucleotides, likewise, are referred to by their commonly accepted single-letter codes.

The term “nucleic acid” or “nucleotide” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions are achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid is one that is separated from at least some of the nucleic acids that normally flank it or other nucleic acids or components (proteins, lipids, etc. . . . ) in a sample. In another example, a polypeptide is purified if it is substantially removed from or concentrated in its natural environment. Methods for purification and isolation of nucleic acids and proteins are documented methodologies. Embodiments of “substantially” include at least 20%, at least 40%, at least 50%, at least 75%, at least 85%, at least 90%, at least 95%, or at least 99%.

The terms “treatment,” “treating,” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. In general, methods of the invention involve treating diseases referred to as cancer and in particular prostate cancer may be applied to a variety of different types of cancer by utilizing combinations of compounds such as tyrosine kinase receptor inhibitors which are known to bind to the receptor site. “Treatment” as used herein covers any treatment of such a disease in a mammal, particularly a human, and includes:

(a) preventing and/or diagnosing the disease in a subject which may be predisposed to the disease which has not yet been diagnosed as having it;

(b) inhibiting the disease, i.e. arresting its development; and/or

(c) relieving the disease, i.e. causing regression of the disease.

The invention is directed towards treating patients with prostate cancer and is particular directed towards treating particular types of prostate cancer which are not generally treatable with normal surgical methods. More specifically, “treatment” is intended, in preferred circumstances, to mean providing a therapeutically detectable and beneficial effect on a patient suffering from cancer and in particular prostate cancer.

### Examples of Pharmaceutical Compositions and Methods of Administration

Pharmaceutical compositions are formulated using one or more physiologically acceptable carriers including excipients and auxiliaries which facilitate processing of the active agents into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include an IGF-1 receptor inhibitor and a pharmaceutically acceptable diluent(s), excipient(s), or carrier(s). In addition, an IGF-1 receptor inhibitor is optionally administered as pharmaceutical compositions in which they are mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of an IGF-1 receptor inhibitor with other chemical components, such as carriers, stabilizers, diluents, dispersing agents, suspending agents, thickening agents, and/or excipients. The pharmaceutical composition facilitates administration of an IGF-1 receptor inhibitor to an organism. In practicing the methods of treatment or use provided herein, therapeutically effective amounts of an IGF-1 receptor inhibitor are administered in a pharmaceutical composition to a mammal having a condition, disease, or disorder to be treated. Preferably, the mammal is a human. A therapeutically effective amount varies depending on the severity and stage of the condition, the age and relative health of the subject, the potency of the IGF-1 receptor inhibitor used and other factors. Antibodies are optionally used singly or in combination with one or more therapeutic agents as components of mixtures.

The pharmaceutical formulations described herein are optionally administered to a subject by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

In some embodiments pharmaceutical compositions comprise an IGF-1 receptor inhibitor, as an active ingredient in free-acid or free-base form, or in a pharmaceutically acceptable salt form. In addition, the methods and pharmaceutical compositions described herein comprise the use of N-oxides, crystalline forms (also known as polymorphs), as well as active metabolites of an IGF-1 receptor inhibitor having the same type of activity. In some situations, an IGF-1 receptor inhibitor exist as tautomers. All tautomers are included within the scope of the agents presented herein. Additionally, in some embodiments, an IGF-1 receptor inhibitor exists in unsolvated as well as solvated forms with pharmaceutically acceptable solvents such as water, ethanol, and the like. The solvated forms of an IGF-1 receptor inhibitor presented herein are also considered to be disclosed herein.

“Carrier materials” include any commonly used excipients in pharmaceutics and should be selected on the basis of compatibility with agents disclosed herein, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include an IGF-1 receptor inhibitor, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use are optionally obtained by mixing one or more solid excipients with an IGF-1 receptor inhibitor, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active agent doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of an IGF-1 receptor inhibitor are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including an IGF-1 receptor inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which includes an IGF-1 receptor inhibitor, are optionally further formulated to provide a controlled release of an IGF-1 receptor inhibitor. Controlled release refers to the release of an IGF-1 receptor inhibitor from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include an IGF-1 receptor inhibitor, are delivered using a pulsatile dosage form. A pulsatile dosage form is capable of providing one or more immediate release pulses at predetermined time points after a controlled lag time or at specific sites. Pulsatile dosage forms including the formulations described herein, which include an IGF-1 receptor inhibitor, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to an IGF-1 receptor inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further include a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, an IGF-1 receptor inhibitor is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of an IGF-1 receptor inhibitor and a suitable powder base such as lactose or starch.

Buccal formulations that include an IGF-1 receptor inhibitor include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of an IGF-1 receptor inhibitor, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of an IGF-1 receptor inhibitor is administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of at least one agent that inhibits IGF-1 receptor; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a formulation comprising an inhibitor of IGF-1 receptor employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery of a formulation. The rate of absorption is optionally slowed by using rate-controlling membranes or by trapping an active agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing a formulation comprising an IGF-1 receptor inhibitor optionally with carriers, optionally a rate controlling barrier to deliver the formulation to the skin of the host at a controlled and predetermined rate over a prolonged period of time, and means to secure the device to the skin.

Formulations that include an inhibitor to IGF-1 receptor suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, an inhibitor to IGF-1 receptor is optionally formulated in aqueous solutions, preferably in physiologically compatible buffers such as Hank's solution, Ringer's solution, or physiological saline buffer. For transmucosal administration, penetrants appropriate to the barrier to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of an IGF-1 receptor inhibitor in water soluble form. Additionally, suspensions of an IGF-1 receptor inhibitor are optionally prepared as appropriate oily injection suspensions.

In some embodiments, an inhibitor to IGF-1 receptor is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

An IGF-1 receptor inhibitor is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

Examples of Methods of Dosing and Treatment Regimens

A formulation comprising an IGF-1 receptor inhibitor for is optionally used in the preparation of medicaments for the prophylactic and/or therapeutic treatment of prostate cancer that would benefit, at least in part, from amelioration. In addition, a method for treating any of the diseases or conditions described herein in a subject in need of such treatment, involves administration of pharmaceutical compositions containing an IGF-1 receptor inhibitor as described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In the case wherein the patient's condition does not improve, upon the doctor's discretion the administration of the IGF-1 receptor inhibitor is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of an IGF-1 receptor inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical composition described herein is in unit dosage forms suitable for single administration of precise dosages. In unit dosage form, the formulation is divided into unit doses containing appropriate quantities of an IGF-1 receptor inhibitor. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

As an example of dosing a male patient weighing approximately 70 kg may be given a dose of NDGA in a range of from 50 to 250 mg/day. Those doses may be provided over a period of time such as providing the doses daily over 90 days. The foregoing ranges are merely suggestive, as the number of variables in regard to an individual treatment regime is large, and considerable excursions from these recommended values are not uncommon. Such dosages are optionally altered depending on a number of variables, not limited to the activity of the IGF-1 receptor inhibitor used, the disease or condition to be treated, the mode of administration, the requirements of the individual subject, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. An IGF-1 receptor inhibitor exhibiting high therapeutic indices is preferred. The data obtained from cell culture assays and animal studies are optionally used in formulating a range of dosage for use in human. The dosage of such an IGF-1 inhibitor lies preferably within a range of circulating concentrations that include the ED50 with minimal toxicity. The dosage optionally varies within this range depending upon the dosage form employed and the route of administration utilized.

### Combination Treatments

IGF-1 receptor compositions described herein are also optionally used in combination with other therapeutic reagents that are selected for their therapeutic value for the condition to be treated. In general, the compositions described herein and, in embodiments where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

In certain instances, it is appropriate to administer an IGF-1 receptor inhibitor composition as described herein in combination with another therapeutic agent. In some instances, for example, an anti-cancer agent may be administered in combination with the IGF-1 receptor inhibitor. Such anti-cancer agents may include at least one cytotoxic or chemotherapeutic agent with the formulation. The cytotoxic or chemotherapeutic agents may include alkylating agents or agents with an alkylating action, such as cyclophosphamide (CTX; e.g. CYTOXAN®), chlorambucil (CHL; e.g. LEUKERAN®), cisplatin (CisP; e.g. PLATINOL®) busulfan (e.g. MYLERAN®), melphalan, carmustine (BCNU), streptozotocin, triethylenemelamine (TEM), mitomycin C, and the like; anti-metabolites, such as methotrexate (MTX), etoposide (VP16; e.g. VEPESID®), 6-mercaptopurine (6 MP), 6-thiocguanine (6TG), cytarabine (Ara-C), 5-fluorouracil (5-FU), capecitabine (e.g. XELODA.®), dacarbazine (DTIC), and the like; antibiotics, such as actinomycin D, doxorubicin (DXR; e.g. ADRIAMYCIN®), daunorubicin (daunomycin), bleomycin, mithramycin and the like; alkaloids, such as vinca alkaloids such as vincristine (VCR), vinblastine, and the like; and other antitumor agents, such as paclitaxel (e.g. TAXOL®) and pactitaxel derivatives, the cytostatic agents, glucocorticoids such as dexamethasone (DEX; e.g. DECADRON®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. ETHYOL®), dactinomycin, mechlorethamine (nitrogen mustard), streptozocin, cyclophosphamide, lomustine (CCNU), doxorubicin lipo (e.g. DOXIL®), gemcitabine (e.g. GEMZAR®), daunorubicin lipo (e.g. DAUNOXOME®), procarbazine, mitomycin, docetaxel (e.g. TAXOTERE®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil.

By way of example only, if one of the side effects experienced by a patient upon receiving an IGF-1 receptor inhibitor composition as described herein is nausea, then it is appropriate to administer an anti-nausea agent in combination with the initial therapeutic agent. In any case, regardless of the disease, disorder or condition being treated, the overall benefit experienced by the patient is either simply additive of the two therapeutic agents or the patient experiences a synergistic benefit.

Therapeutically-effective dosages vary when the drugs are used in treatment combinations. Methods for experimentally determining therapeutically-effective dosages of drugs and other agents for use in combination treatment regimens are documented methodologies. One example of such a method is the use of metronomic dosing, i.e., providing more frequent, lower doses in order to minimize toxic side effects. Combination treatment further includes periodic treatments that start and stop at various times to assist with the clinical management of the patient.

In any case, the multiple therapeutic agents (one of which is an IGF-1 receptor as described herein) are administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents; the use of multiple therapeutic combinations is also envisioned.

It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also be administered sequentially, with either therapeutic agent being administered by a regimen calling for two-step administration. The two-step administration regimen optionally calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentrations are optionally used to determine the optimal dose interval.

In addition, an IGF-1 inhibitor is optionally used in combination with procedures that provide additional or synergistic benefit to the patient. By way of example only, patients are expected to find therapeutic and/or prophylactic benefit in the methods described herein, wherein pharmaceutical compositions of an IGF-1 receptor inhibitor and/or combinations with other therapeutics are combined with genetic testing to determine whether that individual is a carrier of a mutant gene that is correlated with certain diseases or conditions, or will benefit from said therapy.

An IGF-1 receptor inhibitor and the additional therapy(ies) are optionally administered before, during or after the occurrence of a disease or condition, and the timing of administering the composition containing an IGF-1 receptor inhibitor varies in some embodiments. Thus, for example, an IGF-1 receptor inhibitor is used as a prophylactic and is administered continuously to subjects with a propensity to develop conditions or diseases in order to prevent the occurrence of the disease or condition. An IGF-1 receptor inhibitor and compositions are optionally administered to a subject during or as soon as possible after the onset of the symptoms. The administration of the agents are optionally initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration is optionally via any route practical, such as, for example, an intravenous injection, a bolus injection, infusion over 5 minutes to about 5 hours, a pill, a capsule, transdermal patch, buccal delivery, and the like, or combination thereof. An IGF-1 receptor inhibitor is preferably administered as soon as is practicable after the onset of a disease or condition is detected or suspected, and for a length of time necessary for the treatment of the disease, such as, for example, from about 1 month to about 3 months. The length of treatment optionally varies for each subject, and the length is then determined using the known criteria. For example, an IGF-1 receptor inhibitor or a formulation containing an IGF-1 receptor inhibitor, or combinations thereof, are administered for at least 2 weeks, preferably about 1 month to about 5 years, and more preferably from about 1 month to about 3 years.

While embodiments of the present invention have been shown and described herein, it will be obvious to those skilled in the art that such embodiments are provided by way of example only. Numerous variations, changes, and substitutions will now occur to those skilled in the art without departing from the invention. It should be understood that in some embodiments of the invention various alternatives to the embodiments described herein are employed in practicing the invention.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

NDGA and IGF-1 were from Insmed Corporation (Richmond, Va.). The following were purchased: antibodies against the IGF-1 receptor (C-20), phosphospecific antibodies recognizing phosphotyrosine (PY20), and HRP-conjugated anti-phosphotyrosine antibody (PY20HRP) were from Santa Cruz Biotechnology (Santa Cruz, Calif.); alpha IR3, a monoclonal antibody against the IGF-1 receptor, was from CalBiochem (San Diego, Calif.); phosphospecific antibody pIGF-IR (Y1131) was from Cell Signaling (Beverly, Mass.); methyltrienolone (R1881) was from Perkin Elmer Life Sciences, Inc. (Boston, Mass.) and coated protein A Sepharose CL4B was from Amersham Biosciences (Uppsala, Sweden). Unless specified, all other reagents were from Sigma (St. Louis, Mo.).

### Example 1

### Growth Studies of LAPC-4 Prostate Cancer Cells

LAPC-4 prostate cancer cells were maintained at 37° C., 5% CO₂ in phenol-free RPMI+10% FCS RPMI. Steroid free medium consisted of phenol-free RPMI supplemented with 10% dextran-coated, charcoal-treated serum (10% CDSS RPMI). LAPC-4 cells were incubated in this steroid-free 10% CDSS RPMI for 3 days prior to plating in 96 well plates (5×10³ cells/well). Cells were allowed to adhere overnight and were then treated with androgens and various concentrations of NDGA with DMSO as a vehicle control. The medium with androgens and inhibitors was refreshed on day 3. The plates were harvested on day 7 by inverting the microplate onto paper towels with gentle blotting to remove growth medium without disrupting adherent cells and freezing them at −80° C. for at least 30 minutes. LAPC-4 prostate cancer cell growth was determined using either the CyQuant cell proliferation assay (Molecular Probes, Eugene, Oreg.) or by the BCA assay (Pierce, Rockford, Ill.). Cell proliferation was calculated as the percent of content versus control cells at day 0.

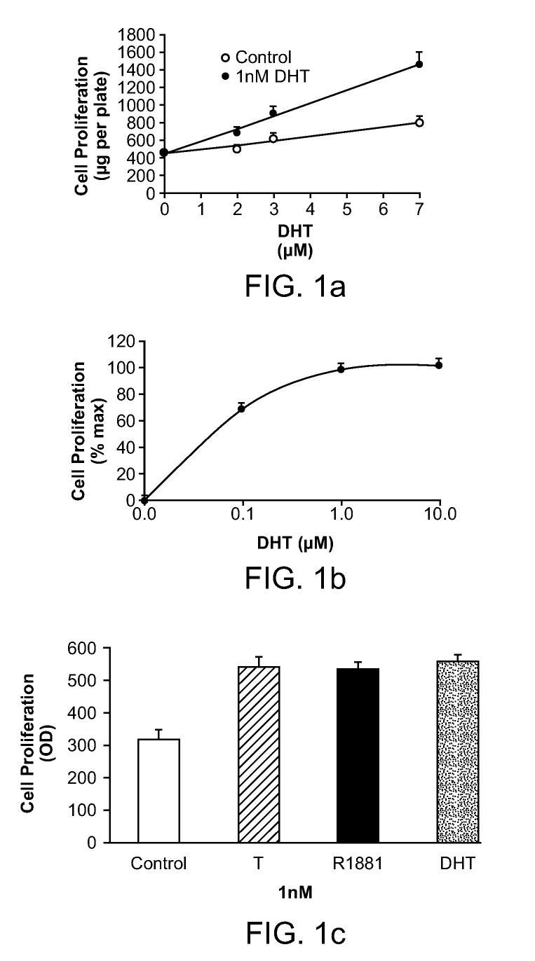

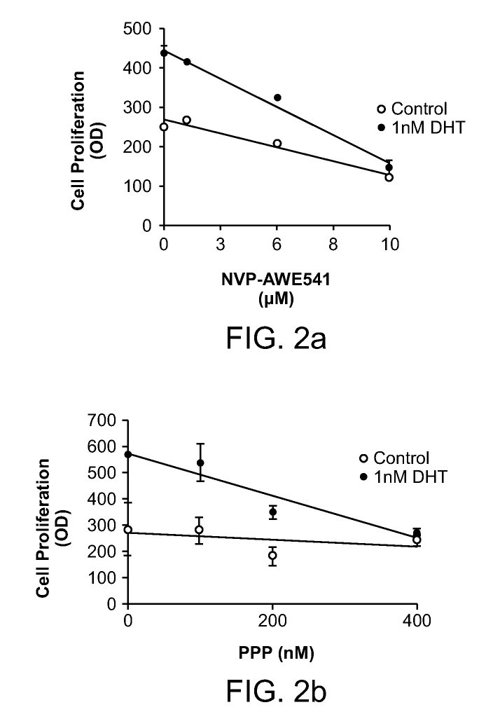

The ability of the androgen, dihydrotestosterone (DHT), to stimulate the proliferation of LAPC-4 cells in culture was evaluated (FIG. 1). Each value shown is the mean+SD for triplicate determinations. In FIGS. 1a and 2b, cell proliferation was measured by BCA, and in FIG. 1c by the CyQuant method. In all future studies, the CyQuant method was employed to measure proliferation.

DHT at 10 nM stimulated cell growth for up to 7 days (FIG. 1a). In 3 separate experiments, at 7 days, the affect of DHT to stimulate growth was 83+8% above control (n=3, mean+SEM). A major effect of DHT was observed at 0.1 nM and maximal effects were observed at 1.0 to 10 mM (FIG. 1b). Two other androgens, testosterone and R1881, both at 1 nM, had similar effects (FIG. 1c).

### Example 2

### Effect of IGF-1 Receptor Inhibitors on DHT-Induced Prostate Cancer Cell Growth

To understand the role of the IGF-1 receptor on the DHT-induced increase in growth, we studied molecules that inhibit this receptor by unrelated mechanisms of action: NVP-AEW541 and picrodopophyllin (PPP). See Garcia-Echeverria et al. (2004) and Gimita et al. (2004). The former blocks ATP binding to the receptor, and the latter blocks substrate phosphorylation. Both agents completely blocked the effect of 1 nM DHT to stimulate proliferation with much smaller effects on non-androgen mediated growth. See FIG. 2; each value is the mean+SD for triplicate determinations. NVP-AEW541 was effective between 1 and 10 μm (FIG. 2a), and PPP was effective between 100 and 400 nM (FIG. 2b). These data support the hypothesis, therefore, that DHT and the IGF-1 receptor may have cooperative functions in stimulating cell growth, and that blocking the function or activity of IGF-1 receptor completely blocks any prostate cancer cell growth mediated by androgen treatment.

### Example 3

### NDGA Inhibitis DHT-Induced Prostate Cancer Cell Growth

LAPC-4 cells were androgen starved, as described above, for 3 days. Cells were plated in 96-well plates in 0.2% agar layer over 0.4% base agar layer as follows: To prepare 0.4% base agar layer, 0.8% agar solution at 37° C. was mixed with 2×10% CDSS RPMI. Cells were harvested and resuspended in culture medium (10% CDSS RPMI). For each well to be plated, 20 μl of 0.8% agar was mixed with 40 μl cells and 20 μl 2×10% CDSS RPMI. When the top layer solidified, 1 nM dihydrotestosterone (DHT) was added in 50 μl of 1×5% CDSS RPMI. NDGA was applied in 50 μl of 1× media the following day. Cells were refreshed on day 3. Cells were grown for 6 days at 37° C., 5% CO₂. Experiments were terminated on day 6, by aspirating the liquid culture and solubilized in 3 M guanidine isothiocyanate at 45° C. for 1 hour. The CyQuant cell proliferation assay was then employed.

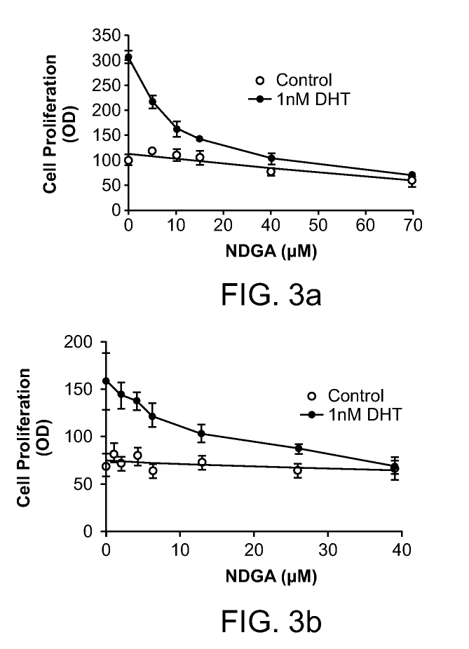

The effect of NDGA on DHT-induced cell proliferation was first evaluated in cells grown on tissue culture plates. See FIG. 3; each value is the mean+SD for triplicate determinations. NDGA, at concentrations between 1 and 30 μM, inhibited androgen stimulation of growth with a smaller effect on non androgen mediated growth (FIG. 3a). In 5 separate experiments, the half-maximal effect of NDGA to inhibit androgen-stimulated growth was 5+1 μM (mean+SEM). Similar inhibition of androgen stimulation of growth was observed when testosterone was used instead of DHT. In addition, a similar effect of NDGA to inhibit the effect of DHT occurred when cells were grown in soft agar indicating that NDGA inhibited anchorage independent growth (FIG. 3b).

### Example 4

### Ligand-Stimulated IGF-1 Receptor Autophosphorylation in LAPC-4 Prostate Cancer Cells

For IGF-1 receptor autophosphorylation, cells were transferred into 10% CDSS RPMI 3 days prior to the experiment. The cells were then plated in 6-well plates in the presence of 1 nM DHT. At the end of either a 1- or 3-day incubation period, cells were serum-starved for 2 hours. NDGA was dissolved in DMSO and diluted with culture medium before being added to cells for 1.5 hours at 37° C. The final concentration of DMSO during the incubation was 0.3%. Cells were then stimulated with 3 nM IGF-I for 10 minutes at 37° C. Reactions were terminated by rapidly aspirating medium and washing cells 3 times with ice cold phosphate-buffered saline (PBS) at 4° C. Cells were harvested and solubilized in 50 mM HEPES, 150 mM NaCl, 1% Triton X-100, 1 mM PMSF, and 2 mM vanadate for 1 hour at 4° C. Protein was determined by BCA assay. IGF-1 receptor autophosphorylation was determined by ELISA. See Youngren et al. (2005). Briefly, 10 μg lysate protein was added to triplicate wells in a 96-well plate coated with monoclonal antibody to the IGF-1 receptor (αIR3; 2 μg/ml), and incubated for 18 hours at 4° C. Plates were washed 5 times, and then HRP-conjugated anti-phosphotyrosine antibody (0.3 ug/ml), diluted in 50 mM HEPES, pH 7.6, 150 mM NaCl, 0.05% Tween-20, 1 mM PMSF, 2 mM vanadate and 1 mg/ml bacitracin, was added for 2 hours at 22° C. Plates were washed 5-times prior to color development with TMB substrate, which was terminated with 1.0 M H3PO₄. Values for receptor autophosphorylation were determined by measuring absorbance at 450 nm.

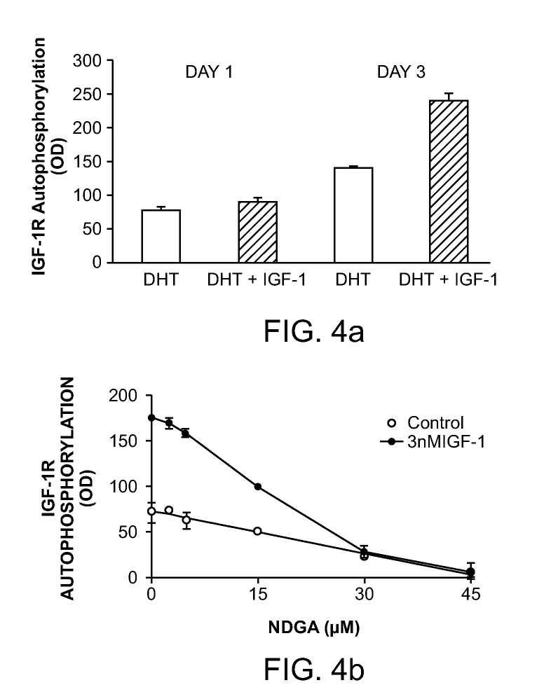

In view of prior observations that NDGA rapidly inhibits ligand-induced activation of the IGF-1 receptor in breast cancer, neuroblastoma, and other cancers, the effect of this agent on the IGF-1 receptor in prostate cancer cells was studied. LAPC-4 cells were incubated with 1 mM DHT for either 1 or 3 days. After 1 day of incubation there was very little stimulation of this IGF-1 receptor autophosphorylation by 10-minute incubation with IGF-1 (FIG. 4a). In contrast, there was a 2-fold stimulation of this function in cells that had been incubated for 3 days with DHT (FIGS. 4a,b). At this time, NDGA inhibited IGF-1 induced IGF-1 receptor autophosphorylation (FIG. 4b) over a concentration range similar to that seen for inhibition of testosterone-mediated growth. In 3 separate experiments, the half-maximal effect of NDGA to inhibit IGF-1 receptor kinase was 12+2 uM (mean+SEM). The observation that IGF-1 activated the IGF-1 receptor after 3 days of incubation with DHT, but not after day 1 of incubation, raised the possibility that NDGA may have had a second mechanism of action; inhibition of androgen-stimulated growth in prostate cancer cells.

### Example 4

### Insulin Receptor/IGF-1 Receptor Studies

Cells were androgen-starved in 10% CDSS RPMI, as described above, prior to their plating in 100 mm dishes with various doses of androgens and/or NDGA. At the end of the incubation period, cells were washed twice with PBS and solubilized with lysis buffer (50 mM HEPES pH 7.6, 150 mM NaCl, 0.1% Triton X-100, 1 mM PMSF, 2 mM Na₃VO4) for 1 hour at 4° C. Immunoprecipitation of IGF-1 receptor was carried out with anti-IGF-1 receptor beta (C-20) coated protein A Sepharose CL4B from 250 μg of cell lysates. The immunoprecipitated samples were run on SDS-PAGE, under reducing condition. Following transfer to nitrocellulose membrane, levels of IGF-1 receptor were assessed by western blot. Following an overnight incubation at 4° C., the membranes were washed 3 times with TBST and then incubated with HRP-conjugated anti-rabbit IgG diluted 1:50,000 in the same blocking buffer for 90 minutes at room temperature. After washing, blots were incubated with Super Signal (Pierce Chemicals, Rockford, Ill.), and exposed to film.

Level of insulin receptor was determined in 15 μg of total protein run on SDS-PAGE under reducing conditions, transferred to nitrocellulose membrane, and analyzed by probing with an anti-insulin receptor antibody CT-3, diluted 1:1000 in 1% milk, 1% BSA in TBST at 4° C. overnight. The secondary anti-mouse IgG, diluted 1:2000 in the same blocking buffer, was applied for 90 minutes at room temperature. Blots were incubated with SuperSignal, and exposed to film.

PCR was conducted in triplicate with 20 μl reaction volumes of Taqman buffer (Applied Biosystems PCR buffer; 20% glycerol, 2.5% gelatin, 60 nM Rox as a passive reference), 5.5 mM MgCl₂, 0.5 mM each primer, 0.2 μM each deoxynucleotide triphosphate (dNTP), 200 nM probe, and 0.025 unit/μl AmpliTaq Gold (Applied Biosystems, CA) with 5 ng cDNA. A large master mix of the above-mentioned components (minus the primers, probe and cDNA) was made for each experiment and aliquoted into individual tubes, one for each cDNA sample. cDNA was then added to the aliquoted master mix. The master mix with cDNA was aliquoted into a 384-well plate, with nine wells used for each cDNA sample. The primers and probes were mixed together and added to the master mix and cDNA in the 384-well plate. PCR was conducted on the ABI 7900HT (Applied Biosystems, CA) using the following cycle parameters: 1 cycle of 95° C. for 10 minutes, 40 cycles of 95° C. for 15 seconds, 60° C. for 1 minute. Analysis was carried out using the SDS software (version 2.3) supplied with the ABI 7900HT. The Ct values for each set of three reactions were averaged for all subsequent calculations. PCR primer and TaqMan probe sequences were either synthesized by Integrated DNA Technologies (Coralville, Iowa) or purchased from Applied Biosystems (Foster City, Calif.). The sequences were as follows:

| H. Cyclophilin | | |
|---|---|---|
| Forward | TCTCAAATCAGAATGGGACAGGT | (SEQ ID NO: 1) |
| Reverse | TGAGAACCGTTTGTGTTGCG | (SEQ ID NO: 2) |
| Probe | 5′-Fam-TTCCATTACAAGCATGATCGGGAGGGT-bhq-3′ | (SEQ ID NO: 3) |
| IGF-1R | | |
| Forward | GAGATCTTGTACATTCGCACCAAT | (SEQ ID NO: 4) |
| Reverse | TTAACTGAGAAGAGGAGTTCGATGCT | (SEQ ID NO: 5) |
| Probe | 5′-Fam-CTTCAGTTCCTTCCATTCCCTTGGXX-bhq1-3′ | (SEQ ID NO: 6) |

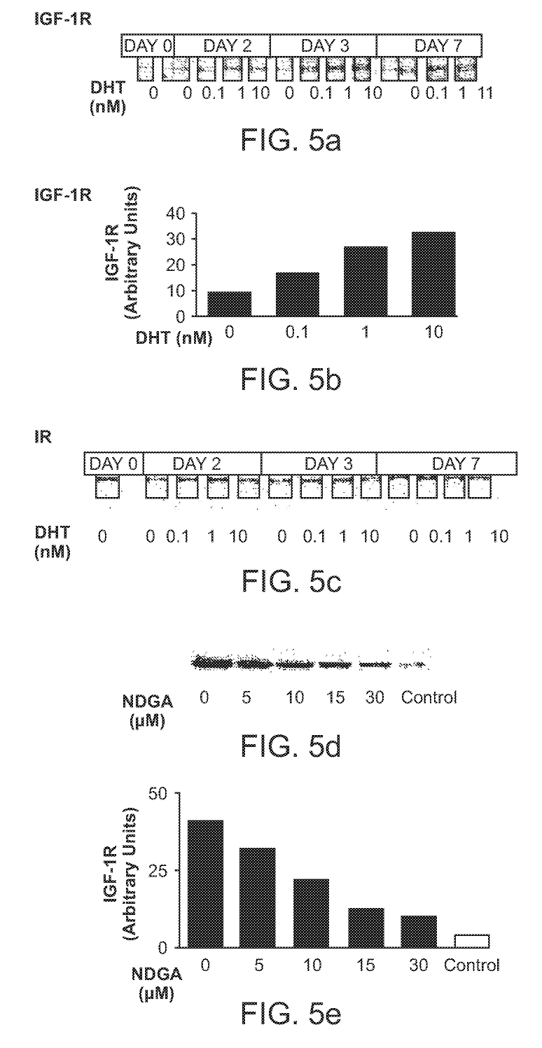

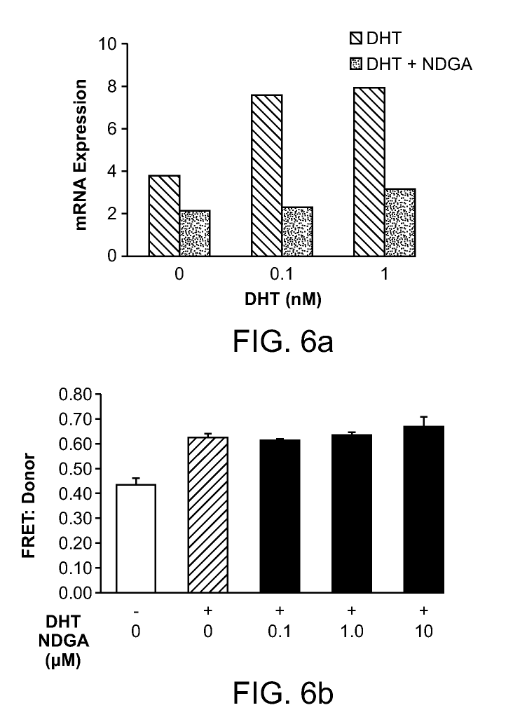

IGF-1 receptor may play a role in the formation and growth of prostate cancer cells. Baserga (1995); Pandini et al. (2005). Further, in prostate cancer cell lines other than LAPC-4, androgens have been shown to increase the expression and function of the IGF-1 receptor. Pandini et al. (2005); Fan et al. (2007). Accordingly, the effect of DHT on the expression of the IGF-1 receptor in LAPC-4 cells was measured. Western blot analyses indicated that DHT markedly increased the content of the IGF-1 receptor (FIG. 5a). An effect was observed after 2 days of incubation and was near-maximal after 3 days of incubation. As with stimulation of proliferation, a significant effect of DHT was observed at 0.1 nM and maximal effects were observed at 1.0 to 10 nM (FIG. 5b). Additionally, an increase in IGF-1 receptor mRNA content was also observed. IGF-1 receptor gene expression was measured by quantitative PCR. DHT was shown to maximally increase IGF-1 receptor gene expression at 0.1 nM (FIG. 6a).

In contrast to the DHT-induced increase of the IGF-1 receptor, there was no change in the content of the closely related insulin receptor (IR) by DHT (FIG. 5c).

Cells were also incubated for 3 days with nM DHT in the absence and presence of NDGA (FIG. 5d). IGF-1 receptor levels were increased by DHT, and this increase was progressively inhibited by increasing concentrations of NDGA from 5 to 15 μM. In 3 separate experiments, the effects of NDGA to inhibit DHT-induced IGF-1 receptor content was 11+2 μM (mean+SEM) (FIG. 5e). Additionally, at 10 μM, NDGA was shown to completely inhibit the effect of DHT to increase the expression of IGF-1 receptor (FIG. 6a).

### Example 5

### NDGA Does Not Inhibit DHT-Induced Changes in Androgen Receptor Conformation

Though IGF-1 receptor transcription is stimulated by androgens, there is evidence that the IGF-1 gene itself is not directly regulated by AR, but rather it is an indirect effect involving new protein synthesis and perhaps Src and MEK1. See Wu et al. (2005). We predicted therefore that NDGA is able to inhibit androgen-induced IGF-1 receptor expression without directly interfering with AR activation. To determine if NDGA interferes with AR activity, we employed an assay that utilizes fluorescence resonance energy transfer (FRET) to measure the confirmation change induced by DHT. Schaufele et al. (2005).

FRET assays were performed as described previously. Briefly, cells stably expressing a CFP-AR-YFP fusion (CAR^{y}) were transferred to black, clear-bottomed 96-well plates along with DHT and NDGA. The cells were fixed in 4% paraformaldehyde and read in PBS on a monochronometer-based fluorescence plate reader (Safire, Tecan, Inc., NC). Each plate contained untransfected, positive, and negative controls. FRET:donor ratios were calculated following background subtraction and correction for acceptor (YFP) contribution to the FRET signal.

Conformation change is a proximal step in the AR activation pathway, which is likely insensitive to the secondary effects of cross-talk between AR and IGF signaling. Using a LAPC-4 cell line expressing the AR FRET reporter, it was observed that NDGA had no effect on the AR conformation change induced by DHT (FIG. 6b). Instead, the FRET analysis of the AR suggests that the NDGA effect on AR action occurs after androgen-induced conformational changes in the AR. These results, thus, suggest that NDGA functions to inhibit IGF-1 receptor expression at a step distal to initial AR activation. In addition, the results also suggest another potential mechanism of NDGA inhibition is through the attenuation of androgen stimulation of IGF-1 receptor expression.

Prior studies from Nickerson and colleagues have demonstrated that tumor growth in xenographic mice bearing LAPC-4 tumor is associated with the expression of both the IGF-1 receptor and its ligand, IGF-1. Nickerson et al. (2001). Pandini and colleagues have reported that androgen stimulation of LNCaP cells results in an increase in expression of the IGF-1 receptor mRNA. Pandini et al. (2005). This effect appeared to be indirect as it was blocked by inhibition of protein synthesis and inhibitors of Src and MEK1, a nonreceptor tyrosine kinase and a dual tyrosine/threonine kinase respectively. Fan et al. have also reported that androgens upregulate the IGF-1 receptor mRNA expression. Fan et al. (2007). Moreover, Fan et al. have reported that: 1) the nuclear factor, Foxol, inhibits AR action; and 2) IGF-1 signaling phosphorylates and inactivates Foxol leading to enhanced AR function. Thus, they propose a positive feedback loop between AR signaling and IGF-1 receptor signaling. Id. The present series of experiments confirm that androgens influence the expression of the IGF-1 receptor in LAPC-4 cells and form the basis for the effect of NDGA on androgen stimulated tumor growth. The combined effects of increased IGF-1 receptor expression in response to androgen stimulation and the potential for a cooperative effect on tumor growth by the AR and IGF-1 receptor make IGF-1 receptor inhibition an attractive clinical strategy therefore in patients with androgen dependent prostate cancer as well as in those with castration-resistant disease.

Clinically, targeting an alternative (or cooperative) pathway to conventional androgen signaling provides several possibilities for providing clinical benefit to patients. The first is that by attenuating androgen signaling in tumor cells without utilizing androgen deprivation therapy, it may delay or reduce the duration of androgen deprivation and its attendant toxicities of osteoporosis, increased risk of cerebrovascular accidents and myocardial infarction, hot flashes, and loss of libido. The second is that targeting the IGF receptor in the setting of androgen deprivation may improve outcomes by either prolonging the sensitivity of the tumor to the androgen deprivation therapy, increasing the proportion of cells within a tumor compartment that undergo apoptosis in response to a therapy, or attenuate one of the signals that is implicated in the emergence of castration resistant therapy. Finally, such therapies may be utilized as secondary therapies in combination with secondary androgen deprivation treatments such as adrenal androgen inhibitors or as monotherapy. Preliminary clinical evaluation of NDGA in patients with both androgen-dependent and androgen-independent prostate cancer has been performed, and has demonstrate reasonable safety and early evidence of clinical effects. Ryan et al. (2008). Notably, as might be predicted by the present series of experiments, modest attenuating effects on the rate of rise of PSA as well as modest declines in PSA were observed occurred in patients with non-castrate levels of testosterone and a rising PSA as their only manifestation of disease.

### REFERENCE LIST

(1) Nieto M, Finn S, Loda M, Hahn W C. Prostate cancer: Re-focusing on androgen receptor signaling. Int J Biochem Cell Biol 2007; 39(9):1562-8.
(2) Scher H I, Sawyers C L. Biology of progressive, castration-resistant prostate cancer: directed therapies targeting the androgen-receptor signaling axis. J Clin Oncol 2005 Nov. 10; 23(32):8253-61.
(3) Baserga R. The insulin-like growth factor I receptor: a key to tumor growth? Cancer Res 1995 Jan. 15; 55(2):249-52.
(4) Burfeind P, Chemicky C L, Rininsland F, Ilan J. Antisense RNA to the type I insulin-like growth factor receptor suppresses tumor growth and prevents invasion by rat prostate cancer cells in vivo. Proc Natl Acad Sci USA 1996; Vol 93, Iss 14:7263-8.
(5) Nickerson T, Chang F, Lorimer D, Smeekens S P, Sawyers C L, Pollak M. In vivo progression of LAPC-9 and LNCaP prostate cancer models to androgen independence is associated with increased expression of insulin-like growth factor I (IGF-I) and IGF-I receptor (IGF-IR) 1. Cancer Res 2001 Aug. 15; 61(16):6276-80.
(6) Pollak M. Insulin-like growth factors and prostate cancer 115. Epidemiol Rev 2001; 23(1):59-66.
(7) LeRoith D, Roberts C T, Jr. The insulin-like growth factor system and cancer 1. Cancer Lett 2003 Jun. 10; 195(2):127-37.
(8) Rubin R, Baserga R. Insulin-like growth factor-I receptor. Its role in cell proliferation, apoptosis, and tumorigenicity. Lab Invest 1995 September; 73(3):311-31.
(9) Sachdev D, Yee D. Disrupting insulin-like growth factor signaling as a potential cancer therapy. Mol Cancer Ther 2007 January; 6(1):1-12.
(10) Yu H, Rohan T. Role of the insulin-like growth factor family in cancer development and progression. J Natl Cancer Inst 2000 Sep. 20; 92(18):1472-89.
(11) Chan J M, Stampfer M J, Giovannucci E, Gann P H, Ma J, Wilkinson P, Hennekens C H, Pollak M. Plasma insulin-like growth factor-I and prostate cancer risk: a prospective study 59. Science 1998 Jan. 23; 279(5350):563-6.
(12) Wolk A, Mantzoros C S, Andersson S O, Bergstrom R, Signorello L B, Lagiou P, Adami H O, Trichopoulos D. Insulin-like growth factor 1 and prostate cancer risk: a population-based, case-control study. J Natl Cancer Inst 1998 Jun. 17; 90(12):911-5.
(13) Khandwala H M, McCutcheon I E, Flyvbjerg A, Friend K E. The effects of insulin-like growth factors on tumorigenesis and neoplastic growth 19. Endocr Rev 2000 June; 21(3):215-44.
(14) Roberts C T, Jr. Insulin-like growth factor I receptor regulation in prostate carcinoma 62. Growth Horm IGF Res 2000 April; 10 Suppl A:S20-S21.
(15) Waller C, Gisvold O. Phytochemical investigation of Larrea divaricata. Journal of the American Pharmaceutical Association 1945; 34:78-86.
(16) Blecha J E, Anderson M O, Chow J M, Guevarra C C, Pender C, Penaranda C, Zavodovskaya M, Youngren J F, Berkman C E. Inhibition of IGF-1R and lipoxygenase by nordihydroguaiaretic acid (NDGA) analogs 1. Bioorg Med Chem Lett 2007 Jul. 15; 17(14):4026-9.
(17) Youngren J F, Gable K, Penaranda C, Maddux B A, Zavodovskaya M, Lobo M, Campbell M, Kerner J, Goldfine I D. Nordihydroguaiaretic acid (NDGA) inhibits the IGF-1 and c-erbB2/HER2/neu receptors and suppresses growth in breast cancer cells 3. Breast Cancer Res Treat 2005 November; 94(1):37-46.
(18) Zavodovskaya M, Campbell M J, Maddux B A, Shiry L, Allan G, Hodges L, Kushner P, Kerner J A, Youngren J F, Goldfine I D. Nordihydroguaiaretic acid (NDGA), an inhibitor of the HER2 and IGF-1 receptor tyrosine kinases, blocks the growth of HER2-overexpressing human breast cancer cells 1. J Cell Biochem 2007 Jun. 11.
(19) Meyer G E, Chesler L, Liu D, Gable K, Maddux B A, Goldenberg D D, Youngren J F, Goldfine I D, Weiss W A, Matthay K K, Rosenthal S M. Nordihydroguaiaretic acid inhibits insulin-like growth factor signaling, growth, and survival in human neuroblastoma cells. J Cell Biochem 2007 May 7.
(20) Ryan C J, Haqq C M, Simko J, Nonaka D F, Chan J M, Weinberg V, Small E J, Goldfine I D. Expression of insulin-like growth factor-1 receptor in local and metastatic prostate cancer 1. Urol Oncol 2007 March; 25(2):134-40.
(21) Craft N, Shostak Y, Carey M, Sawyers C L. A mechanism for hormone-independent prostate cancer through modulation of androgen receptor signaling by the HER-2/neu tyrosine kinase 1. Nat Med 1999 March; 5(3):280-5.
(22) Ngo T H, Barnard R J, Cohen P, Freedland S, Tran C, deGregorio F, Elshimali Y I, Heber D, Aronson W J. Effect of isocaloric low-fat diet on human LAPC-4 prostate cancer xenografts in severe combined immunodeficient mice and the insulin-like growth factor axis 6. Clin Cancer Res 2003 July; 9(7):2734-43.
(23) Schaufele F, Carbonell X, Guerbadot M, Bomgraeber S, Chapman M S, Ma A A, Miner J N, Diamond M I. The structural basis of androgen receptor activation: intramolecular and intermolecular amino-carboxy interactions 4. Proc Natl Acad Sci USA 2005 Jul. 12; 102(28):9802-7.
(24) Garcia-Echeverria C, Pearson M A, Marti A, Meyer T, Mestan J, Zimmermann J, Gao J, Brueggen J, Capraro H G, Cozens R, Evans D B, Fabbro D, Furet P, Porta D G, Liebetanz J, Martiny-Baron G, Ruetz S, Hofmann F. In vivo antitumor activity of NVP-AEW541-A novel, potent, and selective inhibitor of the IGF-IR kinase. Cancer Cell 2004 March; 5(3):231-9.
(25) Gimita A, Gimita L, del P F, Bartolazzi A, Larsson 0, Axelson M. Cyclolignans as inhibitors of the insulin-like growth factor-1 receptor and malignant cell growth 24. Cancer Res 2004 Jan. 1; 64(1):236-42.
(26) Pandini G, Mineo R, Frasca F, Roberts C T, Jr., Marcelli M, Vigneri R, Belfiore A. Androgens up-regulate the insulin-like growth factor-I receptor in prostate cancer cells 1. Cancer Res 2005 Mar. 1; 65(5):1849-57.
(27) Fan W, Yanase T, Morinaga H, Okabe T, Nomura M, Daitoku H, Fukamizu A, Kato S, Takayanagi R, Nawata H. Insulin-like growth factor 1/insulin signaling activates androgen signaling through direct interactions of Foxol with androgen receptor 66. J Biol Chem 2007 Mar. 9; 282(10):7329-38.
(28) Wu J D, Odman A, Higgins L M, Haugk K, Vessella R, Ludwig D L, Plymate S R. In vivo effects of the human type I insulin-like growth factor receptor antibody A12 on androgen-dependent and androgen-independent xenograft human prostate tumors 1. Clin Cancer Res 2005 Apr. 15; 11(8):3065-74.
(29) Ryan C J, Harzstark A H, Youngren J F, Rosenberg J, Lin A, Claros C, Goldfine I D, Kemer J, Small E J. A pilot dose-escalation study of the effects of nordihydroguareacetic acid on hormone and prostate specific antigen levels in patients with relapsed prostate cancer 1. BJU Int 2008 February; 101(4):436-9.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

What is claimed is:

1. A method of treating a human afflicted with prostate cancer, comprising:
administering to a human patient a therapeutically effective amount of a formulation comprising a pharmaceutically acceptable carrier, and an IGF-1 receptor inhibitor; and
allowing the formulation to act on the human and treat the prostate cancer.

2. The method of claim 1, wherein the human is afflicted with androgen non-responsive prostate cancer.

3. The method of claim 1, wherein the human is afflicted with androgen-responsive prostate cancer.

4. The method of claim 1, wherein the IGF-1 receptor inhibitor is selected from the group consisting of a small molecule inhibitor, an antisense oligonucleotide and an antibody.

5. The method of claim 4, wherein the small molecule inhibitor is selected from the group consisting of NDGA, NVP-AEW541 and picropodophyllin.

6. The method of claim 1, wherein the formulation further comprises an androgenic hormone blocking agent.

7. The method of claim 1, wherein the formulation further comprises an agent selected from the group consisting of an LHRH analog, an LHRH antagonist, an antiandrogen, an estrogen, and ketoconazole.

8. The method of claim 1, wherein the formulation further comprises a compound selected from the group consisting of cyclophosphamide and chlorambucil.

9. The method of claim 1, wherein the formulation further comprises:
an agent that inhibits nonreceptor-tyrosine kinases.

10. The method of claim 9, wherein the agent is selected from the group consisting of dasatinib, AZDO530, AP23846, PP2 and UCS15A.

11. The method of claim 1, wherein the formulation further comprises:
a therapeutically effective amount of meso-nordihydroguaiaretic acid (NDGA).

12. The method of claim 11, wherein the formulation further comprises:
an NDGA solvent.

13. The method of claim 12, wherein the solvent is DMSO.

14. The method of claim 1, wherein the formulation further comprises an adrenal androgen inhibitor.

15. The method of claim 1, further comprising:
diagnosing the patient as having metastatic prostate cancer.

16. A method of treating prostate cancer in a human, comprising the steps of:
diagnosing a human patient as having prostate cancer;
determining if the prostate cancer is responsive to androgen therapy;
administering to a human patient a therapeutically effective amount of a formulation comprising a pharmaceutically acceptable carrier, and an IGF-1 receptor inhibitor; and
allowing the formulation to act on the human and treat the prostate cancer.

17. The method of claim 16, further comprising:
administering an androgenic hormone blocking agent; and
allowing the agent to treat the prostate cancer.

18. The method of claim 17, wherein the agent is selected from the group consisting of an LHRH analog, an LHRH antagonist, an antiandrogen, an estrogen, and ketoconazole.

19. The method of claim 18, further comprising:
administering cyclophosphamide to the human patient; and
allowing the cyclophosphamide to treat the prostate cancer.

20. The method of claim 18, further comprising:
administering chlorambucil to the human patient; and
allowing the chlorambucil to treat the prostate cancer.
